(19) United States
(12) Patent Application Publication
(10) Pub. No.: US 2014/0199690 A1
(43) Pub. Date: Jul. 17, 2014

(54) MATERIALS AND METHODS FOR HIGH-THROUGHPUT DETERMINATION OF GENOME-WIDE DNA METHYLATION PROFILE

(71) Applicants: Cunyou Zhao, Hong Kong (CN); Hong Xue, Hong Kong (CN)

(72) Inventors: Cunyou Zhao, Hong Kong (CN); Hong Xue, Hong Kong (CN)

(73) Assignee: THE HONG KONG UNIVERSITY OF SCIENCE AND TECHNOLOGY, Hong Kong (CN)

(21) Appl. No.: 13/743,008

(22) Filed: Jan. 16, 2013

(51) Int. Cl. C12Q 1/68

(52) U.S. Cl. CPC C12Q 1/68
USPC 435/6.11

(57) ABSTRACT

The present invention provides materials and methods for rapid and sensitive determination of global methylation profile of genomic DNA. In one embodiment, the present invention provides the fluorescence polarization (FP) based measurement of DNA methylation (FPDM) assay, wherein the FPDM assay comprises restriction digestion of DNA molecules using a pair of methyl-sensitive and methyl-insensitive restriction endonuclease enzymes, polymerase chain extension of digested DNA molecules via the incorporation of fluorescently labeled dNTP(s), and analysis via fluorescence polarization techniques.

### BACKGROUND OF THE INVENTION

Methylation of cytosine in CpG dinucleotides, a central epigenetic modification in mammalian genomes, controls tissue-specific gene expression, genomic imprinting, chromosome stabilizing, and cellular differentiation. Perturbation of normal DNA methylation is involved in diseases including cancer and psychotic disorders such as schizophrenia. Techniques for rapid determination of DNA methylation status are useful in areas of biotechnology and medicine.

A variety of techniques are available for evaluating changes in genome-wide cytosine methylation based on non-bisulfite conversion methods, including quantitation of methyl-cytosines by reversed phase high performance liquid chromatography (RPHPLC), HPLC-mass spectrometry (HPLC-MS), high performance capillary electrophoresis (HPCE), methyl-C antibody, and methyl-sensitive restriction enzymes.

The methyl-sensitive HpaII and methyl-insensitive MspI isoschizomer pair are most widely used for screening DNA methylation status of the approximately 2.3 million CCGG sites in the human genome. The target sequence for both HpaII and MspI enzymes is CCGG; however, only MspI, but not HpaII, cleaves the methylated Cm5CGG. Therefore, the fraction of unmethylated sites, defined by the HpaII/MspI cleavage ratio, can be determined by cytosine extension analysis with radioactive-labeled dCTP or biotinylated dCTP and pyrosequencing.

Existing techniques for determination of genome-wide cytosine methylation status require the use of a large amount of genomic DNA, radioactive-labeling, and/or time consuming multi-step processing. Therefore, there is a need for improved techniques for determination of genome-wide DNA methylation status.

### BRIEF SUMMARY OF THE INVENTION

The present invention provides materials and methods for rapid and sensitive determination of global methylation profile of genomic DNA. In one embodiment, the present invention provides the fluorescence polarization (FP) based measurement of DNA methylation (FPDM) assay that comprises restriction digestion of DNA molecules using a pair of methyl-sensitive and methyl-insensitive restriction endonuclease enzymes, chain extension of digested DNA molecules via the incorporation of fluorescently labeled dNTP(s) (e.g., dCTPs), and analysis via fluorescence polarization techniques.

In one embodiment, the method for determining methylation profile of cytosine in CpG dinucleotides in DNA of a sample comprises or consists essentially of:

providing a sample containing DNA molecules and obtaining a first sub-sample and a second sub-sample from the sample;

digesting DNA molecules in the first sub-sample with a methyl-sensitive restriction endonuclease that is HpaII, thereby yielding HpaII-digested DNA molecules;

subjecting the HpaII-digested DNA molecules in the first sub-sample to a polymerase chain extension reaction, thereby incorporating fluorescently-labeled dCTPs into the HpaII-digested DNA molecules to generate a first fluorescent signal;

digesting DNA molecules in the second sub-sample with a methyl-insensitive restriction endonuclease that is MspI, thereby yielding MspI-digested DNA molecules;

subjecting the MspI-digested DNA molecules in the second sub-sample to a polymerase chain extension reaction, thereby incorporating fluorescently-labeled dCTPs into the MspI-digested DNA molecules to generate a second fluorescent signal; and determining the methylation profile of cytosine in CpG dinucleotides in DNA of the sample based on the first fluorescent signal and the second fluorescent signal.

In one embodiment, the present invention provides a method for determining methylation profile of cytosine in CpG dinucleotides in a gene of interest by quantitative polymerase chain reaction, wherein the gene of interest comprises at least one CpG dinucleotide methylation site recognized by HpaII.

The present invention also provides kits for determining the methylation profile of one or more nucleotides at one or more DNA methylation sites in DNA (such as genomic DNA) within the genome of a eukaryotic cell or a population of cells. In one embodiment, the kit comprises one or more of the following reagents: a methyl-sensitive restriction endonuclease and a methyl-insensitive restriction endonuclease recognizing the same DNA methylation site comprising a nucleotide capable of being methylated; dNTPs, wherein one or more dNTPs are fluorescently-labeled; DNA polymerase molecules; and buffers.

In certain embodiments, materials and methods of the present invention can be used for analyzing tissue-specific gene expression, genomic imprinting, chromosome stabilizing, cellular differentiation, λ-chromosomal inactivation, RNAi silencing, transcription regulation, aging, polymorphisms, and carcinogenesis of eukaryotic cells or subjects. In one embodiment, materials and methods of the present invention can be used to detect hypermethylation or hypomethylation of a gene or a regulatory region.

### BRIEF DESCRIPTION OF THE DRAWINGS

FIG. 1 illustrates the principle of the fluorescence polarization (FP) based measurement of DNA methylation (FPDM) assay. In one embodiment, the methyl-sensitive HpaII and methyl-insensitive MspI isoschizomer pair for determining the global methylation status of CpG dinucleotides.

FIG. 2 shows robustness of FPDM. (A) Unmethylated and 100% methylated λ DNAs are mixed to produce a 25% methylated DNA preparation, and varying amounts of 2.4-21.6 ng of this preparation are assayed by FPDM. (B) Application of FPDM assay to human genomic DNA from HEK293T cells. In either (A) or (B), the % methylation is obtained from the FP readings of the MspI restricted DNA (filled triangles with standard errors indicated by bars) and HpaII restricted DNA (open triangles with standard errors indicated by bars) after correcting for blank reading in the absence of DNA; the dashed regression line representing % methylation obtained with different amounts of DNA (represented by open circles) is calculated using GraphPad Prism V5. All the data points represent averages from 2-3 independent experiments each with duplications.

FIG. 3 shows accuracy of FPDM. (A) FPDM assay of lambda DNA. Unmethylated and 100% methylated λ DNAs are mixed to produce preparations with different percentile methylations, and 15 ng of each preparation is assayed by FPDM. The x-axis indicates the known %-methylation in the preparation, and y-axis the FPDM estimated % (open triangles with standard errors indicated by bars): y=0% corresponds to the blank-corrected FP value of the unmethylated λ DNA, and y=100% to that of fully methylated λ DNA. (B) FPDM assay of 255-bp DNA. Unmethylated and 100% methylated 255-bp DNAs are mixed to produce preparations with different percentile methylations, and 300 ng of each preparation is assayed by FPDM. The x-axis indicates the known %-methylation in the preparation, and y-axis the FPDM-estimated %: y=0% corresponds to the blank-corrected FP value given by unmethylated 255-bp DNA, and y=100% corresponds to that given by 100% methylated 255-bp DNA. All the data points represent averages from 2-3 independent experiments each with duplications.

FIG. 4 shows validation of FPDM results by gel electrophoresis. (A) Gel-electrophoresis analysis of 255-bp DNA. Lanes from left to right: GeneRuler 100-bp DNA ladder, unrestricted 255-bp DNA, HpaII-restricted 255-bp DNA with 0%, 20%, 40%, 60%, 80% or 100% methylation, and MspI-restricted 255-bp DNA. Arrows mark positions of four bands, which represent from top to bottom: unrestricted 255-bp DNA, nonspecific PCR-product, 156-bp and 99-bp of enzyme-restricted fragments. (B) Regression line between FPDM-determined % methylation (with standard errors indicated by vertical bars) and electrophoresis-determined % methylation (with standard errors indicated by horizontal bars) of 255-bp DNA. All the data points represent averages from 2-3 independent experiments each with duplicate measurements.

FIG. 5 shows analysis of 5-aza-dC induced hypomethylation. 100 ng of DNAs from cultures of HEK293T cells after treatment with 0, 5, or 10 μM 5-aza-dC for 3 days are analyzed by FPDM. The % methylations relative to untreated control are shown in columns with standard errors represented by bars. Significant difference between treated cells and untreated control is indicated as *(P<0.05) or ** (P<0.01) based on two-tailed t-test calculated using GraphPad Prism V5. All the data points represent averages from 2-3 independent experiments each with duplications.

### BRIEF DESCRIPTION OF THE SEQUENCES

SEQ ID NO: 1 is a primer sequence useful according to the present invention.

SEQ ID NO: 2 is a primer sequence useful according to the present invention.

### DETAILED DISCLOSURE OF THE INVENTION

The present invention provides materials and methods for rapid and sensitive determination of global methylation profile of genomic DNA. In one embodiment, the present invention provides the fluorescence polarization (FP) based measurement of DNA methylation (FPDM) assay that comprises restriction digestion of DNA molecules using a pair of methyl-sensitive and methyl-insensitive restriction endonuclease enzymes, polymerase chain extension of digested DNA molecules via the incorporation of fluorescently labeled dCTP, and analysis via fluorescence polarization techniques. Also provided are kits for determining global methylation profile of genomic DNA.

In one embodiment, the present invention provides a method for determining global methylation profile of CpG dinucleotides, wherein the method comprises HpaII/MspI restriction cleavage, chain extension with the use of fluorescent-dCMP, and fluorescence polarization (FP) techniques.

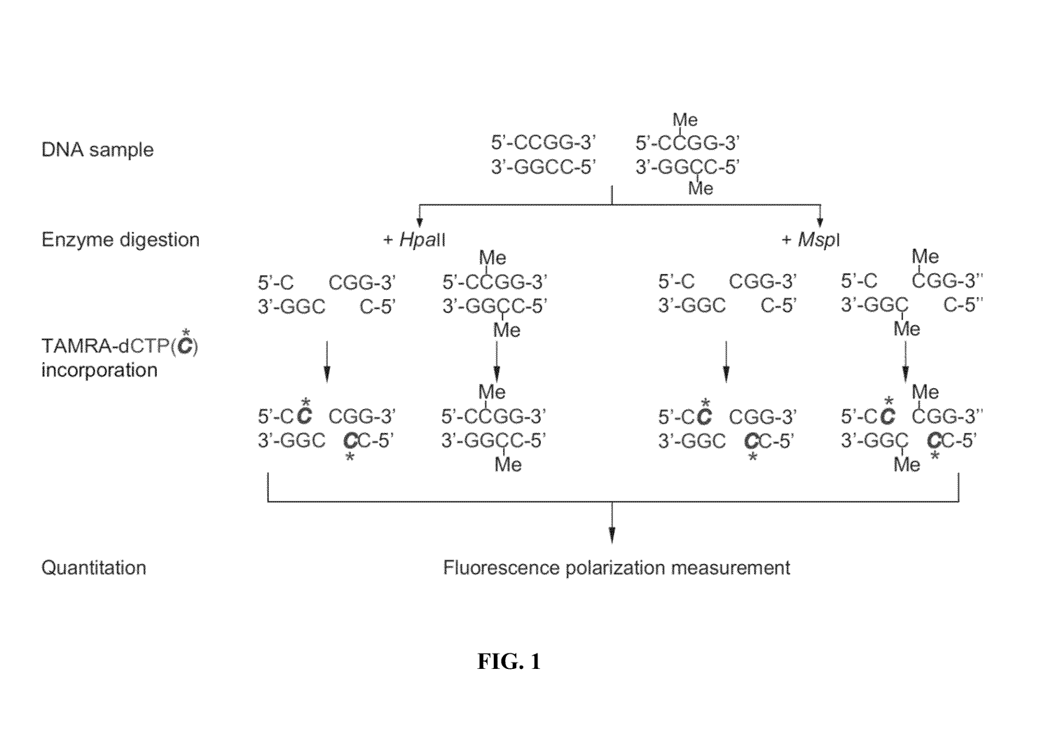

The methyl-sensitive HpaII and methyl-insensitive MspI are Type II restriction enzymes that recognize and digest the CCGG sequence to generate a CpG overhang at the cleavage-generated 5′-terminus on each strand; HpaII and MspI cleavage products can serve as templates for extension of the complementary strand. As shown in FIG. 1, incubation of the restricted DNA with DNA polymerase and TAMRA-dCTP incorporates a single TAMRA-dCMP residue to each strand, resulting in the incorporation of two TAMRA-dCMP residues per restriction site. When the cytosine of the CpG dinucleotides within the CCGG restriction site is methylated, the resultant Cm5CGG sequence is restricted only by MspI, but not by HpaII. Accordingly, the fraction of methylated Cm5CGG sites in the DNA can be determined as [1-(HpaII/MspI)].

The technique of FP is based on that, when fluorescent molecules in solution are excited with plane polarized light, the emitted light is more polarized than that emitted by the molecules that are stationary throughout the excited state.

In the present invention, the HpaII/MspI restricted DNA molecules carrying incorporated fluorescent-dCMP display relatively little movement during excitation and the emitted light remains highly polarized. In contrast, the un-incorporated fluorescent-dCTP molecules rotate and tumble rapidly, such that the emitted light is more depolarized relative to the excitation plane. Since FP measurements can be performed with the use of sub-micrograms of DNA on 96 or 384-well plates without any post-extension purification steps, the present invention is well suited for high-throughput multi-sample applications in biological and clinical studies.

### Methods for Determining Global Methylation Profile

In one embodiment, the present invention provides a method for determining methylation profile of cytosine in CpG dinucleotides in DNA of a sample, wherein the method comprises or consists essentially of:

providing a sample containing DNA molecules and obtaining a first sub-sample and a second sub-sample from the sample;

digesting DNA molecules in the first sub-sample with a methyl-sensitive restriction endonuclease that is HpaII, thereby yielding HpaII-digested DNA molecules;

subjecting the HpaII-digested DNA molecules in the first sub-sample to a polymerase chain extension reaction, thereby incorporating fluorescently-labeled dCTPs into the HpaII-digested DNA molecules to generate a first fluorescent signal;

digesting DNA molecules in the second sub-sample with a methyl-insensitive restriction endonuclease that is MspI, thereby yielding MspI-digested DNA molecules;

subjecting the MspI-digested DNA molecules in the second sub-sample to a polymerase chain extension reaction, thereby incorporating fluorescently-labeled dCTPs into the MspI-digested DNA molecules to generate a second fluorescent signal; and

determining the methylation profile of cytosine in CpG dinucleotides in DNA of the sample based on the first fluorescent signal and the second fluorescent signal.

In one embodiment, the method for determining methylation profile of cytosine in CpG dinucleotides in DNA of a sample further comprises: subjecting the HpaII-digested DNA molecules in the first sub-sample to a quantitative DNA amplification reaction to determine the methylation profile of cytosine in CpG dinucleotides of a gene of interest containing at least one HpaII site, wherein the DNA amplification reaction amplifies DNA molecules undigested by HpaII.

In one embodiment, the DNA amplification reaction is polymerase chain reaction. The term “digestion” or “restriction digestion,” as used herein, refers to a biochemical process in which DNA molecules and restriction endonuclease enzymes are in contact for a sufficient period of time and under conditions that permit cleavage of substantially all cleavable sites recognized by the particular endonuclease enzyme.

In certain embodiments, the present invention provides a method for determining methylation profile in one or more methylation sites in genomic DNA and/or in genes of interest of eukaryotic cells or subjects. In another embodiment, the present invention provides a method for determining methylation profile in one or more methylation sites.

In certain embodiments, the methylation profile of a DNA methylation site can be the ratio of the methylated form and the non-methylated form of a nucleotide in a methylation site of interest; percentage of the methylated form of a nucleotide in a methylation site of interest; or percentage of the non-methylated form of a nucleotide in a methylation site.

The term “genomic DNA” includes all DNA in a cell, group of cells, or in an organelle of a cell; and includes exogenous DNA such as transgenes introduced into a cell.

In one embodiment, the polymerase chain extension reaction is initiated by incubating the target DNA molecules with a reaction mixture comprising DNA polymerase molecules, fluorescently-labeled deoxynucleotides (e.g., fluorescently-labeled dCTP), and buffer.

DNA polymerase enzymes useful for the polymerase chain extension reaction include, but are not limited to, Escherichia coli DNA polymerase I, Thermus aquaticus (Taq) DNA polymerase, Klenow fragment of E. coli DNA polymerase I, T4 DNA polymerase, T7 DNA polymerase Thermus aquaticus (Taq) DNA polymerase, T5 DNA polymerase, and Phi29 DNA polymerase.

Fluorophores useful for labeling dNTPs include, but are not limited to, 5-carboxyfluorescein (FAM), 2′7′ dimethoxy-4′5′-dichloro-6-carboxyfluorescein-cein (JOI), rhodamine, 6-carboxy-rhodamine (R6G), 5/6-carboxytetramethylrhodamine (TAMRA), 6-carboxy-X-rhodamine (ROX), 4-(4′-dimethylaminophenylazo)benzoic acid (DABCYL), and 5-(2′-aminoethyl)aminonaphthalene-1-sulfonic acid (EDANS).

The term “polynucleotide,” as used herein, refers to a nucleic acid molecule containing a sequence that is greater than about 100 nucleotides in length. The term “oligonucleotide,” as used herein, refers a nucleic acid molecule containing a sequence that is of any length between 3 to 30 nucleotides.

As used herein, the term “primer” refers to an oligonucleotide or polynucleotide that is capable of hybridizing or annealing to another nucleic acid of interest under particular stringency conditions. A primer may occur naturally as in a purified restriction digest or be produced synthetically.

The terms “complementary” or “complementarity” are used in reference to nucleic acids (i.e. a sequence of nucleotides) related by the well-known base-pairing rules that A pairs with T or U and C pairs with G. Complementarity can be “partial” in which only some of the nucleotide bases are matched according to the base pairing rules. “Complete” or “total” complementarity between the nucleic acid strands means that all of the bases are matched according to base-pairing rules.

The term “substantially complementary” is used to describe any primer that can hybridize to either or both strands of the target nucleic acid sequence under stringent conditions. As used herein, stringent conditions for hybridization refers to conditions wherein hybridization is typically carried out overnight at 20-25° C. below the melting temperature (Tm) of the DNA hybrid in 6×SSPE, 5×Denhardt's solution, 0.1% SDS, 0.1 mg/ml denatured DNA. The melting temperature, Tm, is described by the following formula (Beltz et al., 1983):

Tm=81.5C+16.6 Log [Na+]+0.41(%G+C)−0.61(% formamide)−600/length of duplex in base pairs.

Washes are carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

In one embodiment, the present invention provides methods and compositions for determining methylation profile of samples including, but not limited to, blood, urine, serum, lymph, saliva, tear, anal and vaginal secretion, perspiration and semen, cell or tissue, and biopsy samples.

The sample may comprise individual cells, including primary cells (including bacteria), and cell lines, including, but not limited to, tumor cells of all types (including melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoietic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO cells, Cos cells, 923 cells, HeLa cells, WI-38 cells, Weri-1 cells, and MG-63 cells.

In one embodiment, the DNA-containing sample for use in the invention is a biological sample obtained from a subject. A biological or tissue sample can be drawn from any tissue that is susceptible to cancer.

In one embodiment, the method further comprises the step of isolating nucleic acid molecules of interest (such as genomic DNA, or genes of interest) from the sample.

In certain embodiment, the sample comprises eukaryotic cells including, but not limited to, animal, plant, and fungal cells. In certain embodiments, the eukaryotic cells are cells of mammalian species including, but not limited to, humans, apes, chimpanzees, orangutans, humans, monkeys; and domesticated and/or laboratory animals such as dogs, cats, horses, cattle, pigs, sheep, goats, chickens, mice, rats, guinea pigs, and hamsters.

The term “subject,” as used herein, describes an organism, including mammals such as primates. Mammalian species that can benefit from the subject methods include, but are not limited to, apes, chimpanzees, orangutans, humans, monkeys; and domesticated and/or laboratory animals such as dogs, cats, horses, cattle, pigs, sheep, goats, chickens, mice, rats, guinea pigs, and hamsters. Typically, the subject is a human.

In one embodiment, the present invention provides a method for determining global DNA methylation profile within the genome of a eukaryotic cell or a population of cells.

In one embodiment, the method comprises obtaining a sample of genomic DNA from a eukaryotic cell or a population of cells; digesting genomic DNA of a first sub-sample of the sample with the methyl-sensitive restriction endonuclease enzyme HpaII, and digesting genomic DNA of a second sub-sample of the sample with the methyl-insensitive restriction endonuclease MspI; subjecting the HpaII-digested and the MspI-digested genomic DNA to a polymerase chain extension reaction, thereby incorporating fluorescent-labeled dCTPs into the HpaII-digested and the MspI-digested DNA molecules; calibrating the fluorescent-labeled DNA by measuring its fluorescence-polarization (FP); and determining the fraction of methylated Cm5CGG sites in the genomic DNA in the sample by the FP value of [1−(HpaII/MspI)].

In certain embodiments, the restriction digestion of DNA molecules is performed with no more than 50 U (or any value less than 50 U) HpaII or MspI per ug DNA. In certain embodiments, the restriction digestion reaction is performed at about 37° C. for 12 hrs.

In certain embodiments, 100 ng of the restricted DNA is incubated in 15 ul of reaction buffer comprising (10 mM Tris-HCl, pH about 9.0, 1.5 mM MgCl₂ and 50 mM KCl), 0.75 U Taq DNA polymerase (TaKaRa), 0.56 nM fluorescent TAMRA-dCTP (5-propargylamino-dCTP-5/6-carboxytetramethylrhodamine, Jena Bioscience, USA) at 57.8° C. for 1 hr.

In certain embodiments, the incorporation of TAMRA-dCMP into DNA amplification products is determined by FP using a Wallac VictorTM2 V 1420 Multilabel Counter (PerkinElmer, Boston, USA).

In certain embodiments, the methylation profile of CpG dinucleotides in DNA of a sample is determined based on the FP readings of the HpaII- and MspI-treated samples (after calibration with the blank FP reading).

In one embodiment, the present invention provides a method for determining the methylation profile of a DNA methylation site in DNA of a sample, wherein the DNA methylation site comprises a nucleotide capable of being methylated,

wherein the method comprises or consists essentially of:

providing a sample containing DNA molecules and obtaining a first sub-sample and a second sub-sample of the sample;

digesting DNA molecules of the first sub-sample with a methyl-sensitive restriction endonuclease that recognizes the DNA methylation site comprising the nucleotide capable of being methylated or unmethylated, thereby yielding digested DNA molecules in the first sub-sample;

subjecting the digested DNA molecules in the first sub-sample to a polymerase chain extension reaction, thereby incorporating fluorescently-labeled dNTPs into the digested DNA molecules in the first sub-sample to generate a first fluorescent signal;

digesting DNA molecules of the second subsample with a methyl-insensitive restriction endonuclease that recognizes the DNA methylation site comprising the nucleotide capable of being methylated or unmethylated, thereby yielding digested DNA molecules in the second sub-sample;

subjecting the digested DNA molecules in the second sub-sample to a polymerase chain extension reaction, thereby incorporating fluorescently-labeled dNTPs into the digested DNA molecules in the second sub-sample to generate a second fluorescent signal;

determining the methylation profile of the DNA methylation site of DNA molecules in the sample based on the first fluorescent signal and the second fluorescent signal;

wherein the methyl-sensitive restriction endonuclease and the methyl-insensitive restriction endonuclease recognize the same DNA methylation site comprising the nucleotide capable of being methylated or unmethylated; and

wherein the methyl-sensitive restriction endonuclease cleaves the DNA molecules preferentially if the restriction recognition site is unmethylated, whereas the methyl-insensitive restriction endonuclease cleaves the DNA molecules regardless whether the restriction recognition site is methylated.

In one embodiment, the method for determining methylation profile of a DNA methylation site in DNA of a sample further comprises: subjecting the methyl-sensitive restriction endonuclease-digested DNA molecules in the first sub-sample to a quantitative DNA amplification reaction to determine the methylation profile of cytosine in CpG dinucleotides of a gene of interest, wherein the DNA amplification reaction amplifies the DNA molecules containing at least one methyl-sensitive restriction endonuclease site wherein the DNA molecules are undigested by the methyl-sensitive restriction endonuclease.

In one embodiment, the methyl-sensitive restriction endonuclease cleaves the DNA molecules only when the restriction recognition site is unmethylated, whereas the methyl-insensitive restriction endonuclease cleaves the DNA molecules regardless whether the restriction recognition site is methylated.

In certain embodiments, the present invention can be used to determine the methylation profile of a methylation site selected from CpG, TpG, CpA, CpHpG, or CpHpH, wherein H represents any nucleotide but guanine. In one embodiment, the present invention can be used to determine the methylation profile of CpG dinucleotide.

In one embodiment, one or more dNTPs (e.g., dATP, dCTP, dGTP, dTTP) are fluorescently-labeled, thereby allowing for determination of the methyl-sensitive restriction digestion of methylation site.

Methylation-sensitive and methylation-insensitive restriction endonuclease enzymes are known in the art. Non-limiting examples of methylation-sensitive and methylation-insensitive restriction endonuclease enzymes useful according to the present invention are shown in Table 1. Based on the methylation site of interest, one skilled in the art could readily select appropriate methylation-sensitive and methylation-insensitive restriction endonuclease enzymes to practice the present invention.

TABLE 1

| Enzyme | Sequence | CpG |
|---|---|---|
| AatII | GACGT/C | [symbol] |
| Acc65I | G/GTACC | [symbol] |
| AccI | GT/MKAC | [symbol] |
| Acil | CCGC(−3/−1) | [symbol] |
| AclI | AA/CGTT | [symbol] |
| AcuI | CTGAAG(16/14) | [symbol] |
| AfeI | AGC/GCT | [symbol] |
| AflII | C/TTAAG | [symbol] |
| AflIII | A/CRYGT | [symbol] |
| AgeI | A/CCGGT | [symbol] |
| AgeI-HF ™ | A/CCGGT | [symbol] |
| AgeI-HF ™ RE-Mix ® | A/CCGGT | [symbol] |
| AluI | AG/CT | [symbol] |
| AlwI | GGATC(4/5) | [symbol] |
| AlwNI | CAGNNN/CTG | [symbol] |
| ApaI | GGGCC/C | [symbol] |
| ApaLI | G/TGCAC | [symbol] |
| ApeKI | G/CWGC | [symbol] |
| ApoI | R/AATTY | [symbol] |
| AscI | GG/CGCGCC | [symbol] |
| AscI RE-Mix ® | GG/CGCGCC | [symbol] |
| AseI | AT/TAAT | [symbol] |
| AsiSI | GCGAT/CGC | [symbol] |
| AvaI | C/YCGRG | [symbol] |
| AvaII | G/GWCC | [symbol] |
| AvrII | C/CTAGG | [symbol] |
| BaeGI | GKGCM/C | [symbol] |
| BamHI | G/GATCC | [symbol] |
| BamHI-HF ™ | G/GATCC | [symbol] |
| BanI | G/GYRCC | [symbol] |
| BanII | GRGCY/C | [symbol] |
| BbsI | GAAGAC(2/6) | [symbol] |
| BbvCI | CCTCAGC(−5/−2) | [symbol] |
| BbvI | GCAGC(8/12) | [symbol] |
| BccI | CCATC(4/5) | [symbol] |
| BceAI | ACGGC(12/14) | [symbol] |
| BciVI | GTATCC(6/5) | [symbol] |
| BclI | T/GATCA | [symbol] |
| BcoDI | GTCTC(1/5) | [symbol] |
| BfaI | C/TAG | [symbol] |
| BfuAI | ACCTGC(4/8) | [symbol] |
| BfuCI | /GATC | [symbol] |
| BglII | A/GATCT | [symbol] |
| BlpI | GC/TNAGC | [symbol] |
| BmgBI | CACGTC(−3/−3) | [symbol] |
| BmrI | ACTGGG(5/4) | [symbol] |
| BmtI | GCTAG/C | [symbol] |
| BmtI-HF ™ | GCTAG/C | [symbol] |
| BpmI | CTGGAG(16/14) | [symbol] |
| Bpu10I | CCTNAGC(−5/−2) | [symbol] |
| BpuEI | CTTGAG(16/14) | [symbol] |
| BsaAI | YAC/GTR | [symbol] |
| BsaBI | GATNN/NNATC | [symbol] |
| BsaHI | GR/CGYC | [symbol] |
| BsaI | GGTCTC(1/5) | [symbol] |
| BsaI-HF ™ | GGTCTC(1/5) | [symbol] |
| BsaJI | C/CNNGG | [symbol] |
| BsaWI | W/CCGGW | [symbol] |
| BseRI | GAGGAG(10/8) | [symbol] |
| BseYI | CCCAGC(−5/−1) | [symbol] |
| BsgI | GTGCAG(16/14) | [symbol] |
| BsiEI | CGRY/CG | [symbol] |
| BsiHKAI | GWGCW/C | [symbol] |
| BsiWI | C/GTACG | [symbol] |
| BsmAI | GTCTC(1/5) | [symbol] |
| BsmBI | CGTCTC(1/5) | [symbol] |
| BsmFI | GGGAC(10/14) | [symbol] |
| BsmI | GAATGC(1/−1) | [symbol] |
| BsoBI | C/YCGRG | [symbol] |
| Bsp1286I | GDGCH/C | [symbol] |
| BspCNI | CTCAG(9/7) | [symbol] |
| BspDI | AT/CGAT | [symbol] |
| BspEI | T/CCGGA | [symbol] |
| BspHI | T/CATGA | [symbol] |
| BspMI | ACCTGC(4/8) | [symbol] |
| BspQI | GCTCTTC(1/4) | [symbol] |
| BsrBI | CCGCTC(−3/−3) | [symbol] |
| BsrDI | GCAATG(2/0) | [symbol] |
| BsrFI | R/CCGGY | [symbol] |
| BsrGI | T/GTACA | [symbol] |
| BsrI | ACTGG(1/−1) | [symbol] |
| BssHII | G/CGCGC | [symbol] |
| BssKI | /CCNGG | [symbol] |
| BssSI | CACGAG(−5/−1) | [symbol] |
| BstBI | TT/CGAA | [symbol] |
| BstEII | G/GTNACC | [symbol] |
| BstEII-HF ™ | G/GTNACC | [symbol] |
| BstEII-HF ™ RE-Mix ® | G/GTNACC | [symbol] |
| BstNI | CC/WGG | [symbol] |
| BstUI | CG/CG | [symbol] |
| BstYI | R/GATCY | [symbol] |
| BstZ17I | GTA/TAC | [symbol] |
| Bsu36I | CC/TNAGG | [symbol] |
| BtgI | C/CRYGG | [symbol] |
| BtgZI | GCGATG(10/14) | [symbol] |
| BtsCI | GGATG(2/0) | [symbol] |
| BtsI | GCAGTG(2/0) | [symbol] |
| BtsIMutI | CAGTG(2/0) | — |
| Cac8I | GCN/NGC | [symbol] |
| ClaI | AT/CGAT | [symbol] |
| CviAII | C/ATG | [symbol] |
| CviKI-1 | RG/CY | [symbol] |
| CviQI | G/TAC | [symbol] |
| DdeI | C/TNAG | [symbol] |
| DpnI | GA/TC | [symbol] |
| DpnII | /GATC | [symbol] |
| DraI | TTT/AAA | [symbol] |
| EaeI | Y/GGCCR | [symbol] |
| EagI | C/GGCCG | [symbol] |
| EagI-HF ™ | C/GGCCG | [symbol] |
| EarI | CTCTTC(1/4) | [symbol] |
| EciI | GGCGGA(11/9) | [symbol] |
| Eco53kI | GAG/CTC | — |
| EcoO109I | RG/GNCCY | [symbol] |
| EcoP15I | CAGCAG(25/27) | [symbol] |
| EcoRI | G/AATTC | [symbol] |
| EcoRI-HF ™ RE-Mix ® | G/AATTC | [symbol] |
| EcoRI-HF ™ | G/AATTC | [symbol] |
| EcoRV | GAT/ATC | [symbol] |
| EcoRV-HF ™ RE-Mix ® | GAT/ATC | [symbol] |
| EcoRV-HF ™ | GAT/ATC | [symbol] |
| FatI | /CATG | [symbol] |
| FauI | CCCGC(4/6) | [symbol] |
| Fnu4HI | GC/NGC | [symbol] |
| FokI | GGATG(9/13) | [symbol] |
| FseI | GGCCGG/CC | [symbol] |
| FspEI | CC(12/16) | [symbol] |
| FspI | TGC/GCA | [symbol] |
| HaeII | RGCGC/Y | [symbol] |
| HaeIII | GG/CC | [symbol] |
| HgaI | GACGC(5/10) | [symbol] |
| HhaI | GCG/C | [symbol] |
| HincII | GTY/RAC | [symbol] |
| HindIII | A/AGCTT | [symbol] |
| HindIII-HF ™ | A/AGCTT | [symbol] |
| HinfI | G/ANTC | [symbol] |
| HinP1I | G/CGC | [symbol] |
| HpaI | GTT/AAC | [symbol] |
| HpaII | C/CGG | [symbol] |
| HphI | GGTGA(8/7) | [symbol] |
| Hpy166II | GTN/NAC | [symbol] |
| Hpy188I | TCN/GA | [symbol] |
| Hpy188III | TC/NNGA | [symbol] |
| Hpy99I | CGWCG/ | [symbol] |
| HpyAV | CCTTC(6/5) | [symbol] |
| HpyCH4III | ACN/GT | [symbol] |
| HpyCH4IV | A/CGT | [symbol] |
| HpyCH4V | TG/CA | [symbol] |
| KasI | G/GCGCC | [symbol] |
| KpnI | GGTAC/C | [symbol] |
| KpnI-HF ™ | GGTAC/C | [symbol] |
| KpnI-HF ™ RE-Mix ® | GGTAC/C | [symbol] |
| LpnPI | CCDG(10/14) | [symbol] |
| MboI | /GATC | [symbol] |
| MboII | GAAGA(8/7) | [symbol] |
| MfeI | C/AATTG | [symbol] |
| MfeI-HF ™ RE-Mix ® | C/AATTG | [symbol] |
| MfeI-HF ™ | C/AATTG | [symbol] |
| MluCI | /AATT | [symbol] |
| MluI | A/CGCGT | [symbol] |
| MlyI | GAGTC(5/5) | [symbol] |
| MmeI | TCCRAC(20/18) | [symbol] |
| MnlI | CCTC(7/6) | [symbol] |
| MscI | TGG/CCA | [symbol] |
| MseI | T/TAA | [symbol] |
| MspA1I | CMG/CKG | [symbol] |
| MspI | C/CGG | [symbol] |
| MspJI | CNNR(9/13) | [symbol] |
| NaeI | GCC/GGC | [symbol] |
| NarI | GG/CGCC | [symbol] |
| Nb.BbvCI | CCTCAGC | [symbol] |
| Nb.BsmI | GAATGC | [symbol] |
| Nb.BsrDI | GCAATG | [symbol] |
| Nb.BtsI | GCAGTG | — |
| NciI | CC/SGG | [symbol] |
| NcoI | C/CATGG | [symbol] |
| NcoI-HF ™ RE-Mix ® | C/CATGG | [symbol] |
| NcoI-HF ™ | C/CATGG | [symbol] |
| NdeI | CA/TATG | [symbol] |
| NgoMIV | G/CCGGC | [symbol] |
| NheI | G/CTAGC | [symbol] |
| NheI-HF ™ RE-Mix ® | G/CTAGC | [symbol] |
| NheI-HF ™ | G/CTAGC | [symbol] |
| NlaIII | CATG/ | [symbol] |
| NlaIV | GGN/NCC | [symbol] |
| NmeAIII | GCCGAG(21/19) | [symbol] |
| NotI | GC/GGCCGC | [symbol] |
| NotI-HF ™ RE-Mix ® | GC/GGCCGC | [symbol] |
| NotI-HF ™ | GC/GGCCGC | [symbol] |
| NruI | TCG/CGA | [symbol] |
| NsiI | ATGCA/T | [symbol] |
| NspI | RCATG/Y | [symbol] |
| Nt.AlwI | GGATC(4/−5) | [symbol] |
| Nt.BbvCI | CCTCAGC(−5/−7) | [symbol] |
| Nt.BsmAI | GTCTC(1/−5) | [symbol] |
| Nt.BspQI | GCTCTTC(1/−7) | [symbol] |
| Nt.BstNBI | GAGTC(4/−5) | [symbol] |
| Nt.CviPII | (0/−1)CCD | [symbol] |
| Pad | TTAAT/TAA | [symbol] |
| Pad RE-Mix ® | TTAAT/TAA | [symbol] |
| PaeR7I | C/TCGAG | [symbol] |
| PciI | A/CATGT | [symbol] |
| PflFI | GACN/NNGTC | [symbol] |
| PhoI | GG/CC | [symbol] |
| PleI | GAGTC(4/5) | [symbol] |
| PmeI | GTTT/AAAC | [symbol] |
| PmlI | CAC/GTG | [symbol] |
| PpuMI | RG/GWCCY | [symbol] |
| PsiI | TTA/TAA | [symbol] |
| PspGI | /CCWGG | [symbol] |
| PspOMI | G/GGCCC | [symbol] |
| PspXI | VC/TCGAGB | [symbol] |
| PstI | CTGCA/G | [symbol] |
| PstI-HF ™ | CTGCA/G | [symbol] |
| PvuI | CGAT/CG | [symbol] |
| PvuI-HF ™ | CGAT/CG | [symbol] |
| PvuII | CAG/CTG | [symbol] |
| PvuII-HF ™ | CAG/CTG | [symbol] |
| RsaI | GT/AC | [symbol] |
| RsrII | CG/GWCCG | [symbol] |
| Sad | GAGCT/C | [symbol] |
| SacI-HF ™ | GAGCT/C | [symbol] |
| SacII | CCGC/GG | [symbol] |
| SalI | G/TCGAC | [symbol] |
| SalI-HF ™ RE-Mix ® | G/TCGAC | [symbol] |
| SalI-HF ™ | G/TCGAC | [symbol] |
| SapI | GCTCTTC(1/4) | [symbol] |
| Sau3AI | /GATC | [symbol] |
| Sau96I | G/GNCC | [symbol] |
| SbfI | CCTGCA/GG | [symbol] |
| SbfI-HF ™ | CCTGCA/GG | [symbol] |
| ScaI-HF ™ RE-Mix ® | AGT/ACT | [symbol] |
| ScaI-HF ™ | AGT/ACT | [symbol] |
| ScrFI | CC/NGG | [symbol] |
| SexAI | A/CCWGGT | [symbol] |
| SfaNI | GCATC(5/9) | [symbol] |
| SfcI | C/TRYAG | [symbol] |
| SfoI | GGC/GCC | [symbol] |
| SgrAI | CR/CCGGYG | [symbol] |
| SmaI | CCC/GGG | [symbol] |
| SmlI | C/TYRAG | [symbol] |
| SnaBI | TAC/GTA | [symbol] |
| SpeI | A/CTAGT | [symbol] |
| SpeI RE-Mix ® | A/CTAGT | [symbol] |
| SpeI-HF ™ | A/CTAGT | [symbol] |
| SphI | GCATG/C | [symbol] |
| SphI-HF ™ | GCATG/C | [symbol] |
| SspI | AAT/ATT | [symbol] |
| SspI-HF ™ | AAT/ATT | [symbol] |
| StuI | AGG/CCT | [symbol] |
| StyD4I | /CCNGG | [symbol] |
| StyI | C/CWWGG | [symbol] |
| StyI-HF ™ | C/CWWGG | [symbol] |
| SwaI | ATTT/AAAT | [symbol] |
| Taq^{α}I | T/CGA | [symbol] |
| TfiI | G/AWTC | [symbol] |
| TseI | G/CWGC | [symbol] |
| Tsp45I | /GTSAC | [symbol] |
| TspMI | C/CCGGG | [symbol] |
| Tth111I | GACN/NNGTC | [symbol] |
| XbaI | T/CTAGA | [symbol] |
| XbaI RE-Mix ® | T/CTAGA | [symbol] |
| XhoI | C/TCGAG | [symbol] |
| XhoI RE-Mix ® | C/TCGAG | [symbol] |
| XmaI | C/CCGGG | [symbol] |
| ZraI | GAC/GTC | [symbol] |

Legend [symbol]
Not Sensitive [symbol]
Blocked [symbol] Impaired [symbol]
Blocked by Overlapping [symbol] Impaired by Overlapping [symbol]
Blocked by Some Combinations of Overlapping [symbol] Impaired by Some Combinations of Overlapping

In certain embodiments, methylation-sensitive and methylation-insensitive restriction endonuclease enzymes useful according to the present invention include, but are not limited to, AatII, AciI, AclI, AgeI, AscI, AvaI, BamHI, BsaA1, BsaH1, BsiE, BsiW, BsrF, BssHII, BstBI, BstUI, Cla1, EagI, HaeII, HgaI, HhaI, HinPI, HpaII, MloI, MspI, NaeI, NarI, NotI, NruI, PmlI, SmaI, and SacII.

The term “consisting essentially of,” as used herein, limits the scope of the ingredients and steps to the specified materials or steps and those that do not materially affect the basic and novel characteristic(s) of the present invention, i.e., compositions and methods for determining methylation profile of a methylation site of interest. For instance, by using “consisting essentially of,” the method of the present invention does not contain any unspecified steps materially affect the determination of global methylation profile; such steps include, but are not limited to, non-bisulfite conversion methods, including quantitation of methyl-cytosines reversed phase high performance liquid chromatography (RPHPLC), HPLC-mass spectrometry (HPLC-MS), high performance capillary electrophoresis (HPCE), and methyl-C antibody; radioactive-labeling of dCTP, biotinylated-dCTP, and pyrosequencing; and nucleic acid hybridization. By using the term “consisting essentially of,” the methods of the present invention may comprise additional steps that do not materially affect the determination of global methylation profile; such steps include, but are not limited to, obtaining a sample from a subject.

In one embodiment, the method for determination of methylation profile of the present invention does not comprise one or more of the following steps including, non-bisulfite conversion methods, including quantitation of methyl-cytosines reversed phase high performance liquid chromatography (RPHPLC), HPLC-mass spectrometry (HPLC-MS), high performance capillary electrophoresis (HPCE), and methyl-C antibody; radioactive-labeling of dCTP, biotinylated-dCTP, and pyrosequencing; nucleic acid hybridization; polymerase chain reaction; and DNA methylase reaction.

Kits

The present invention also provides kits for determining the methylation profile of one or more nucleotides at one or more DNA methylation sites in DNA (such as genomic DNA) within the genome of a eukaryotic cell or a population of cells. In one preferred embodiment, the kit can be used in high-throughput assays for sensitive and rapid determination of global DNA methylation profile.

In one embodiment, the kit comprises one or more of the following reagents: a methyl-sensitive restriction endonuclease and a methyl-insensitive restriction endonuclease recognizing the same DNA methylation site comprising a nucleotide capable of being methylated; dNTPs (e.g., dATP, dCTP, dGTP, dTTP), wherein one or more dNTPs are fluorescently-labeled, thereby allowing for determination of the methyl-sensitive restriction digestion of a methylation site; and DNA polymerase for incorporation of fluorescently-labeled dNTPs (e.g., dCTPs).

In one embodiment, the primers do not amplify undigested DNA molecules. In one embodiment, oligonucleotide primers useful according to the present invention are selected from SEQ ID NO:1 and SEQ ID NO:2.

In certain embodiments, the kit further comprises one or more of the reagents selected from buffers (such as Tris-acetate, magnesium acetate, potassium acetate, dithiothreitol), diluents, preservatives, probes, nucleotides, DNA polymerases, and other enzymes, wherein the reagents can be admixed prior to use. The kit can also comprise a solid support such as microtiter multi-well plates, standards, assay diluents, wash buffers, adhesive plate covers, and/or instructions for carrying out a method of the invention using the kit. In certain embodiments, the kits are adapted to contain compartments for two or more of the above-listed components.

### Medical and Industrial Applications

DNA methylation plays a role in a variety of physiological processes including tissue-specific gene expression, genomic imprinting, chromosome stabilizing, cellular differentiation, X-chromosomal inactivation, RNAi silencing, transcription regulation, aging, polymorphisms, and carcinogenesis.

In certain embodiments, materials and methods of the present invention can be used for analyzing tissue-specific gene expression, genomic imprinting, chromosome stabilizing, cellular differentiation, X-chromosomal inactivation, RNAi silencing, transcription regulation, aging, polymorphisms, and carcinogenesis of eukaryotic cells or subjects. In one embodiment, materials and methods of the present invention can be used to detect hypermethylation or hypomethylation of a gene or a regulatory region.

In certain embodiments, the materials and methods of the present invention can be used for diagnosis and prognosis of diseases including, but not limited to, schizophrenia, bipolar disorder, Alzheimer's disease, diabetes, atherosclerosis, cell proliferative disorders including cancers, major psychosis, lupus, and Parkinson's disease.

The term “cell proliferative disorder,” as used herein, refers to malignant as well as non-malignant cell populations which often differ from the surrounding tissue both morphologically and genotypically. In certain embodiments, the cell proliferative disorder is a cancer. In certain embodiments, the materials and methods of the present invention can be used for the diagnosis and/or prognosis of cancers including, but not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas.

In certain embodiments, the materials and methods of the present invention can be used for monitoring for methylation profiles of livestock in relation to environmental conditions (e.g. drought, weather, change in diet, toxins, diseases etc.).

### Materials and Methods

### DNA Preparation

A 255-bp DNA fragment containing a single CCGG MspI or HpaII cleavage site at position 99-102-bp is PCR-amplified from Intron 8 of the type A γ-aminobutyric acid receptor β₂-subunit (GABRB2) gene of human genomic DNA, and corresponding to bp 160,758,740-160,758,994 on Chromosome 5, using the PCR primers of TTCACCGTGTTAGCCAGGATGGT (SEQ ID NO:1) and CCTAATGGGGGAGTTTGAAC (SEQ ID NO:2).

Amplification is performed in a 20 μl reaction mixture containing 10 ng genomic DNA, 75 nM of each of the primers, 50 nM of each dNTP, 1×PCR buffer and 1 U Taq DNA polymerase (TaKaRa, Dalian, China). The PCR reaction conditions are: initial denaturation at 94° C. for 2 min, 35 cycles each of 15 s at 95° C., 30 s at 58° C., and 30 s at 72° C., plus a final extension step at 72° C. for 5 min. After purification using ethanol precipitation as described in Zhao et al. (2006), 1 μg of unmethylated 255-bp amplicons are fully methylated by incubation at 37° C. for 3 hrs in a 20 μl mixture containing 1 U M.SssI methylase (New England BioLabs, USA), 1×NEBuffer 2, and 160 mM Sadenosylmethionine (New England BioLabs), followed by ethanol precipitation to yield the 100% methylated 255-bp DNA. Unmethylated lambda DNA (TaKaRa) is similarly methylated to yield the 100% methylated lambda DNA.

Human genomic DNA is extracted using DNAzol reagent (Invitrogen, USA) from human embryonic kidney HEK293T cells (ATCC, USA) cultured for 3 days in DMEM supplemented with 10% fetal bovine serum, either in the absence or presence of 5-aza-dC (5-aza-2′-deoxycytidine, Sigma-Aldrich, Sweden).

### FPDM Assay

In the assay, 50-500 ng DNA sample is restricted by incubation at 37° C. in a 50 μl reaction mixture containing 10 U/μg DNA of HpaII or MspI (both from TaKaRa), 1× Buffer T (33 mM Tris-acetate, pH 7.9, 10 mM magnesium acetate, 66 mM potassium acetate and 0.5 mM dithiothreitol) and 0.01% BSA either for 3 hrs with the 255-bp DNA, or for 12 hours with lambda or human genomic DNA.

10 μl of the restricted DNA is placed on a 384-well plate in 15 μl chain-extension reaction mixture containing 1×PCR buffer (10 mM Tris-HCl, pH 9.0, 1.5 mM Mg₂Cl₂ and 50 mM KCl), 0.75 U Taq DNA polymerase (TaKaRa), 0.56 nmoles fluorescent TAMRAdCTP (5-propargylamino-dCTP-5/6-carboxytetramethylrhodamine, Jena Bioscience, USA) at 57.8° C. for 1 hr. Incorporation of TAMRA-dCMP into DNA in each well is measured by FP using a Wallac Victor^{TM2} V 1420 Multilabel Counter (PerkinElmer, Boston, USA) without any prior purification of the chain-extended DNA.

After correcting for the blank FP reading, the FP readings of the HpaII- and MspI-treated samples yield the extent of methylation in the sample: % methylation=[1−HpaII/MspI]*100% wherein HpaII/MspI represents the ratio between the blank-corrected FP readings from the HpaII-induced and MspI-induced TAMRA-dCMP incorporations.

### Gel Electrophoresis

HpaII- or MspI-restricted and unrestricted 255-bp DNA is dissolved in 2% EBargarose gel and electrophoresis is performed at 100 V for 20 minutes. Bands are quantitated using Image Lab Software (BioRad, USA) based on the intensities of their UV absorbance.

### EXAMPLE

Following is an example that illustrates embodiments and procedures for practicing the invention. The example should not be construed as limiting. All percentages are by weight and all solvent mixture proportions are by volume unless otherwise noted.

### Example 1

### Fluorescence Polarization (FP) Based Measurement of DNA Methylation (FPDM) Assay

To rapidly determine DNA methylation levels from a large number of biological or clinical samples, an accurate and sensitive method for high-throughput quantification of global methylation of 5′-Cm5CGG-3′ sites in the genome is provided.

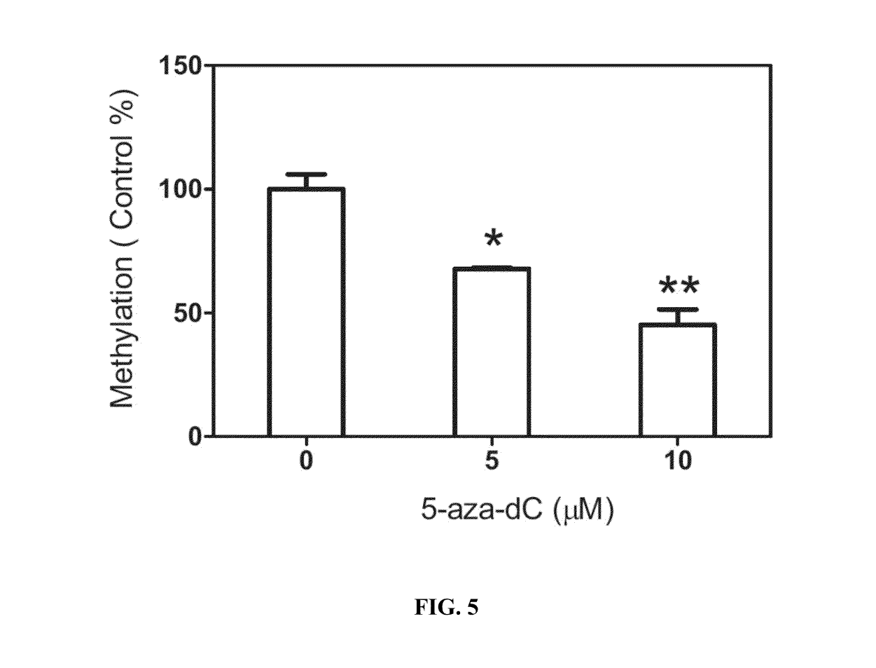

FIG. 1 shows the fluorescence polarization (FP) based measurement of DNA methylation (FPDM) assay of the present invention. In the FPDM assay, the methyl-sensitive HpaII and methyl-insensitive MspI restriction enzymes are employed to achieve DNA cleavage, followed by incorporation of fluorescent dCMP into the enzyme-cleavage products through polymerase chain extension, yielding an FP-ratio between the HpaII- and MspI-restricted preparations as a measurement of methylation profile. The FPDM assay provides stable estimates of methylation level of submicrograms of lambda or human DNA, and of a 255-bp DNA segment containing a single HpaII/MspI restriction site. The results obtained using the FPDM assay are consistent with, and more accurate than, the results obtained using gel electrophoresis. FPDM is also applied to measure dose-dependent DNA hypomethylation in human embryonic kidney 293T cells treated with the DNA-methyltransferase inhibitor 5-aza-dC (FIG. 5).

### Robustness and Accuracy of the FPDM Assay

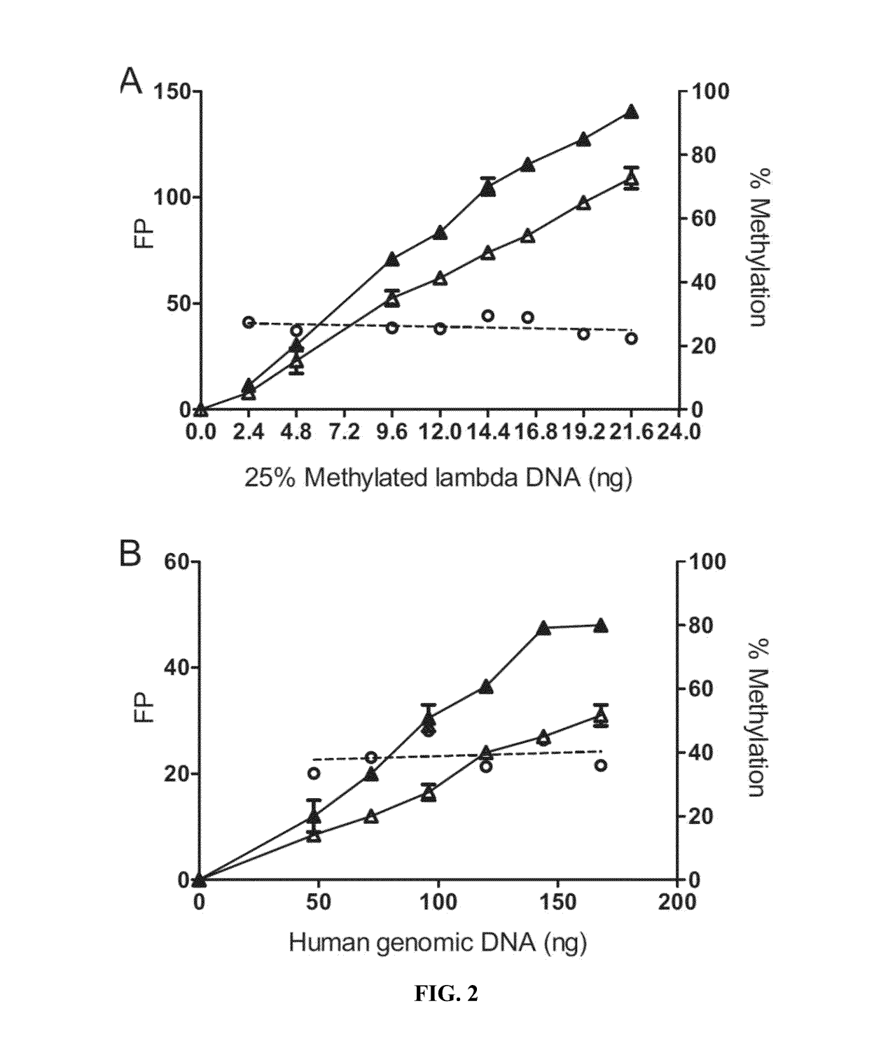

When FPDM assay is applied to varying amounts of 25% methylated lambda DNA (FIG. 2A), the % methylation estimated at 2.4-21.6 ng DNA/assay conformed to a dashed regression line (open circles) with a slight negative slope of −0.1092% ng⁻¹, pointing to a fairly stable % methylation estimate of 25.97±0.88% over this DNA concentration range, in agreement with the expected level of 25% methylation.

When FPDM assay is applied to human genomic DNA (FIG. 2B), an estimated % 40.50±2.33% methylation is provided by the dashed regression line, which also displays a slight negative slope of −0.0212% ng⁻¹ over the concentration range of 48-192 ng DNA/assay.

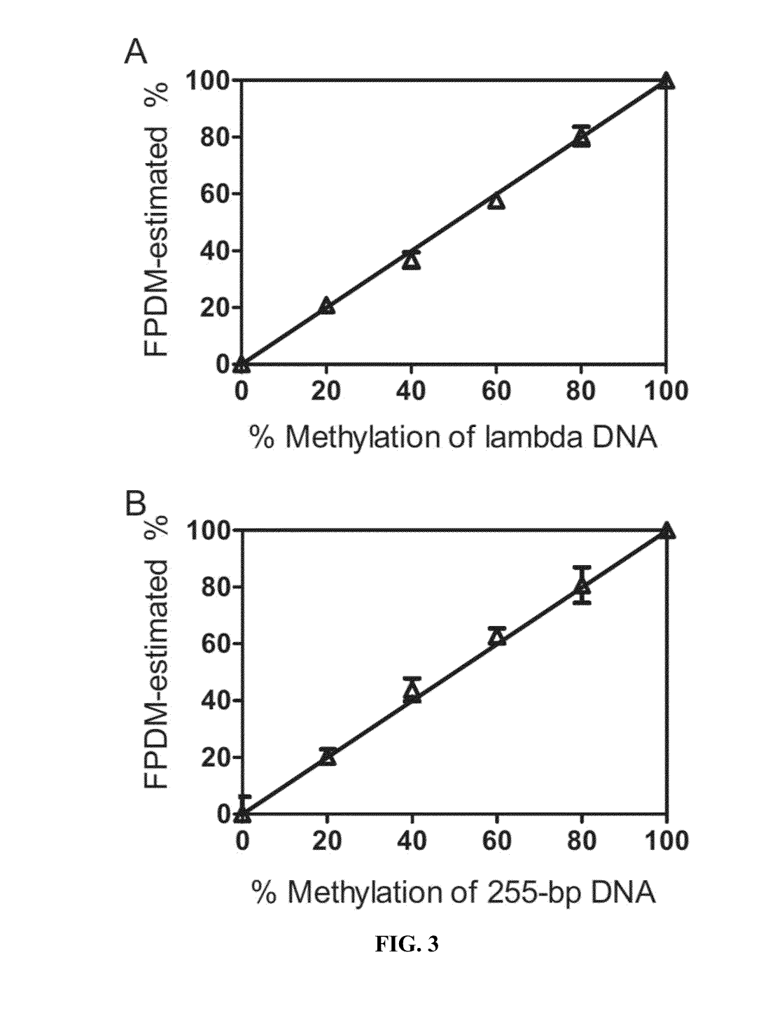

In FIG. 3A, 15 ng of a mixture of methylated and unmethylated lambda DNA in varying proportion is assayed by FPDM. The FPDM-estimated % methylation is 21.88±1.26%, 36.77±2.36%, 57.74±1.86%, and 80.39±3.27%, respectively for the 20%, 40%, 60% and 80% methylated preparations. The results demonstrate the applicability of FPDM over this range of DNA methylation levels.

In FIG. 3B, mixtures each containing 300 ng of methylated and unmethylated forms of a 255-bp DNA fragment (see Materials and Methods) in varying proportions are analyzed by FPDM to yield estimated methylation levels of 20.24±0.94%, 43.79±2.29%, 62.79±1.54% and 80.65±3.63% respectively for the 20%, 40%, 60% and 80% methylated preparations. The results show the applicability of FPDM over this range of DNA methylation levels.

### Validation of the FPDM Assay by Gel Electrophoresis

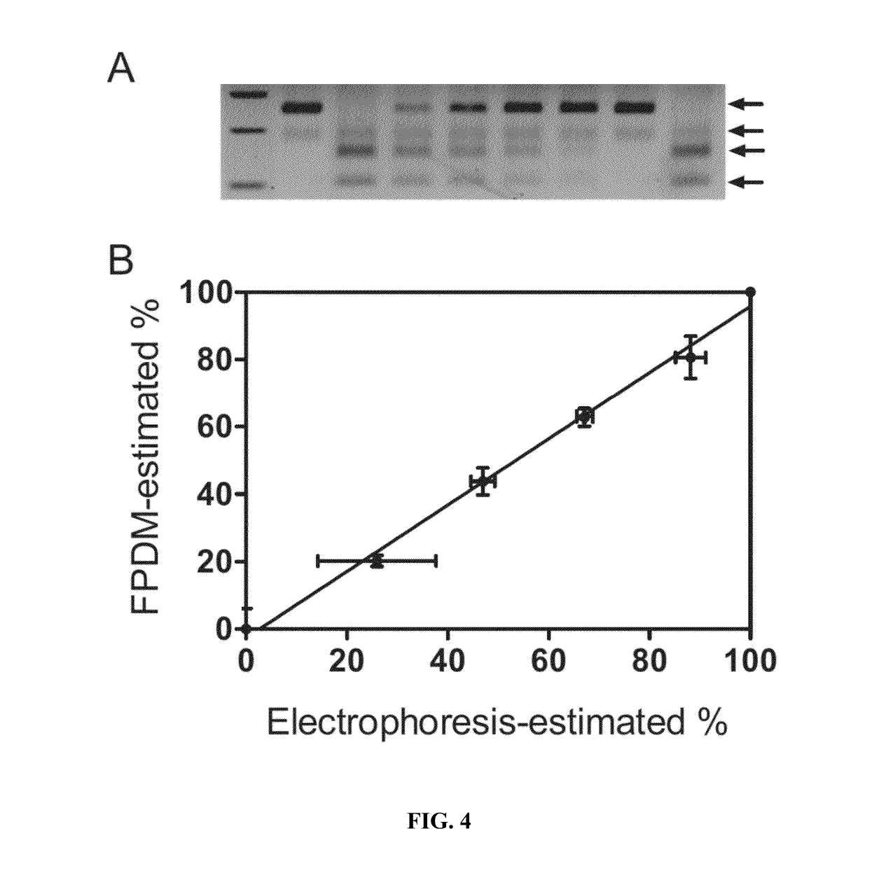

The validity of FPDM is tested using gel electrophoresis analysis. In FIG. 4A, enzyme-restricted and unrestricted 255-bp DNA preparations with different methylation levels are subjected to gel electrophoresis. In each of lanes 3-8, the amount of unrestricted 255-bp DNA is given by the intensity of the top band, and that of HpaII-restricted unmethylated 255-bp DNA is given by the sum of 3rd and 4th bands from the top, corresponding respectively to the 156-bp and 99-bp restriction fragments.

Quantification of the sum of the 156-bp and 99-bp fragments by Image Lab Software yields the total quantity of unmethylated 255-bp DNA cut by HpaII, and in turn the % methylation in the mixture. On this basis, the electrophoresis method yields estimates of 26.00±11.72%, 46.98±2.36%, 67.12±0.14%, and 88.17±3.00% respectively for the 20%, 40%, 60% and 80% methylated preparations. In FIG. 4B the FPDM-estimated % are plotted against electrophoresis-estimated %.

The linear relationship between them (R²=0.99, P<0.001) points to extensive agreement between DNA methylation level determined using the FPDM assay and gel electrophoresis analysis, and therefore, provides valuable cross-validation of the FPDM method by the independent electrophoresis method.

### Analysis of 5-Aza-dC Induced Hypomethylation Status

When HEK293T cells are treated with 5-aza-dC, a known inhibitor of DNA methylation, the cellular DNA methylation measured by FPDM indicates that DNA methylation is inhibited to 67.73±0.53% of control by 5 μM aza-dC, and to 45.16±8.84% of control by 10 μM aza-dC (FIG. 5). These results are consistent with observation of about 42% hypomethylation induced by 5 μM aza-dC relative to the untreated control in murine embryonic fibroblasts cells.

Methylation of cytosine residues in prokaryotic and eukaryotic genomes is an important epigenetic mechanism in gene regulation. In vertebrate DNA, 3% to 5% of cytosine residues are present as 5-methylcytosine, which occurs predominantly at CpG sites. However, low levels of cytosine methylation have also been observed at non-CpG sites such as CpNpG sites.

Restriction enzyme-based methods furnish powerful tools for DNA methylation analysis. The most commonly used isoschizomers in these methods are HpaII/MspI, which recognize CCGG sites. Since these sites account for 8.14% of CpG sites in the human genome, and in general 60-90% of the cytosines in CpG sites are methylated, the methylation status of CCGG sites usefully provides a representative index of global DNA C-methylation level.

In assessing restriction by HpaII relative to MspI, the FPDM procedure avoids the need for radioactivity, or complex protocols such as separation of methylated from unmethylated cytosine by HPCE washings to remove unincorporated biotinylated nucleotide, four-step pyrosequencing, or pre-labeling of PCR amplicons to allow FRET measurement.

Thus, the FPDM assay of the present invention can be readily performed on a 96 or 384-well microtiter plate. As shown in this Example, FPDM has yielded accurate methylation estimates of lambda and human genomic DNA over sub-microgram ranges. Cross-validated by gel electrophoresis, the method has provided a facile high-throughput procedure for measuring global DNA methylation of CCGG sites in DNA preparations as well as in cellular genomes.

All references, including publications, patent applications and patents, cited herein are hereby incorporated by reference to the same extent as if each reference was individually and specifically indicated to be incorporated by reference and was set forth in its entirety herein.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the examples and embodiments described herein are for illustrative purposes only and that various modifications or changes in light thereof will be suggested to persons skilled in the art and are to be included within the spirit and purview of this application.

### REFERENCE

Zhao C, Xu Z, Chen J, Yu Z, Tong K L, Lo W S, et al. Two isoforms of GABAA receptor β2 subunit with different electrophysiological properties: differential expression and genotypical correlations in schizophrenia. Mol Psychiatry 2006; 11:1092-105.

1. A method for determining global quantitation of methylated cytosine in CpG (cytosine-phosphate-guanine) dinucleotides in genomic DNA of a sample, wherein the method comprises:
providing a sample containing genomic DNA molecules and obtaining a first sub-sample and a second sub-sample from the sample;
digesting genomic DNA molecules in the first sub-sample with a methyl-sensitive restriction endonuclease that is HpaII, thereby yielding HpaII-digested DNA molecules with CpG overhangs on each strand;
subjecting a predetermined amount of the HpaII-digested DNA molecules in the first sub-sample to a polymerase chain extension reaction to incorporate fluorescently-labeled dCTPs (cytosine deoxyribonucleoside triphosphate) into the CpG overhangs on each strand of the HpaII-digested DNA molecules to yield HpaII-digested genomic DNA incorporating fluorescently-labeled dCTPs;
determining a first fluorescent signal generated by the HpaII-digested genomic DNA incorporating fluorescently-labeled dCTPs by a fluorescence polarization technique;
digesting genomic DNA molecules in the second sub-sample with a methyl-insensitive restriction endonuclease that is MspI, thereby yielding MspI-digested DNA molecules with CpG overhangs on each strand;
subjecting a predetermined amount of the MspI-digested DNA molecules in the second sub-sample to a polymerase chain extension reaction to incorporate fluorescently-labeled dCTPs into the CpG overhangs on each strand of the MspI-digested DNA molecules to yield MspI-digested genomic DNA incorporating fluorescently-labeled dCTPs;
determining a second fluorescent signal generated by the MspI-digested genomic DNA incorporating fluorescently-labeled dCTPs by a fluorescence polarization technique; and
quantifying methylated cytosine in CpG dinucleotides in genomic DNA of the sample based on the first fluorescent signal and the second fluorescent signal.

2. (canceled)

3. (canceled)

4. The method according to claim 1, wherein the polymerase chain extension reaction is performed using an enzyme selected from Escherichia coli DNA polymerase I, Thermus aquaticus (Taq) DNA polymerase, Klenow fragment of E. coli DNA polymerase I, T4 DNA polymerase, T7 DNA polymerase Thermus aquaticus (Taq) DNA polymerase, T5 DNA polymerase, and Phi29 DNA polymerase.

5. The method according to claim 1, wherein the dCTP is fluorescently labeled using a fluorophore selected from the group consisting of 5-carboxyfluorescein (FAM), 2′7′ dimethoxy-4′5′-dichloro-6-carbosyfjuores-cein (JOI), rhodamine, 6-carboxy-rhodamine (R6G), 5/6-carboxytetramethylrhodamine (TAMRA), 6-carboxy-X-rhodamine (ROX), 4-(4′-dimethylaminophenylazo)benzoic acid (DABCYL), and 5-(2′-aminoethyl)aminonaphthalene-1-sulfonic acid (EDANS).

6. The method according to the claim 1, wherein the sample is a biological sample containing DNA molecules.

7. The method according to claim 6, wherein the biological sample is a blood, urine, serum, lymph, saliva, tear, anal or vaginal secretion, perspiration, semen, cell, tissue, tumor, or biopsy sample.

8. The method according to claim 6, wherein the biological sample comprises eukaryotic cells.

9. The method according to claim 8, wherein the biological sample comprises mammalian cells.

10. (canceled)

11. The method according to claim 1, wherein the methylation profile in the sample is obtained after determining a baseline in the absence of DNA.

12. (canceled)

13. (canceled)

14. A method for quantifying global methylation of DNA methylation sites in genomic DNA of a sample, wherein the DNA methylation sites comprises a nucleotides capable of being methylated, wherein the method comprises:
providing a sample containing genomic DNA molecules and obtaining a first sub-sample and a second sub-sample of the sample;
digesting DNA molecules of the first sub-sample with a methyl-sensitive restriction endonuclease that recognizes the DNA methylation sites comprising nucleotides capable of being unmethylated, thereby yielding digested DNA molecules with overhangs at a cleavage-generated 5′-terminus on each strand in the first sub-sample;
subjecting a predetermined amount of the digested DNA molecules in the first sub-sample to a polymerase chain extension reaction to incorporate fluorescently-labeled dNTPs into the overhangs at the cleavage-generated 5′-terminus on each strand of the digested DNA molecules in the first sub-sample to yield methyl-sensitive-restriction-endonuclease-digested DNA incorporating fluorescently-labeled dNTPs;
determining a first fluorescent signal generated by the methyl-sensitive-restriction-endonuclease-digested DNA incorporating fluorescently-labeled dNTPs by a fluorescence polarization technique; and
digesting DNA molecules of the second subsample with a methyl-insensitive restriction endonuclease that recognizes DNA methylation site comprising nucleotides capable of being methylated or unmethylated, thereby yielding digested DNA molecules with overhangs at a cleavage-generated 5′-terminus on each strand in the second sub-sample;
subjecting a predetermined amount of the digested DNA molecules in the second sub-sample to a polymerase chain extension reaction to incorporate fluorescently-labeled dNTPs into the overhangs at the cleavage-generated 5′-terminus on each strand of the digested DNA molecules in the second sub-sample to yield methyl-insensitive-restriction-endonuclease-digested DNA incorporating fluorescently-labeled dNTPs;
determining a second fluorescent signal generated by the methyl-insensitive-restriction-endonuclease-digested DNA incorporating fluorescently-labeled dNTPs by a fluorescence polarization technique;
quantifying the global methylation of DNA methylation sites of genomic DNA molecules in the sample based on the first fluorescent signal and the second fluorescent signal;
wherein the methyl-sensitive restriction endonuclease and the methyl-insensitive restriction endonuclease recognize the same DNA methylation sites comprising nucleotides capable of being methylated or unmethylated; and
wherein the methyl-sensitive restriction endonuclease cleaves the DNA molecules preferentially if the restriction recognition site is unmethylated, whereas the methyl-insensitive restriction endonuclease cleaves the DNA molecules regardless of whether the restriction recognition site is methylated.

15. The method according to claim 14, wherein the methylation sites are selected from CpG (cytosine-phosphate-guanine), TpG (thymine-phosphate-guanine), CpA (cytosine-phosphate-adenine), CpHpG (cytosine-phosphate-H-phosphate-guanine), or CpHpH (cytosine-phosphate-H-phosphate-H), wherein H represents any nucleotide but guanine.

16. (canceled)

17. A kit for determining the methylation profile of cytosine in CpG dinucleotides in DNA of a sample, wherein the kit comprises a methyl-sensitive restriction endonuclease and a methyl-insensitive restriction endonuclease recognizing the same DNA methylation site comprising a nucleotide capable of being methylated; dNTPs, wherein one or more dNTPs are fluorescently-labeled, thereby allowing for determination of the methyl-sensitive restriction digestion of a methylation site; and DNA polymerase molecules.

18. A kit according to claim 17, wherein the methyl-sensitive restriction endonuclease is HpaII.

19. A kit according to claim 17, wherein the methyl-insensitive restriction endonuclease is MspI.

20. A kit according to claim 17, further comprising fluorescently-labeled dCTPs.


SEQUENCE LISTING (2 sequences)

SEQ ID NO: 1 - DNA, 23 bp, artificial sequence
  the sequence is synthesized
ttcaccgtgttagccaggatggt

SEQ ID NO: 2 - DNA, 20 bp, artificial sequence
  the sequence is synthesized
cctaatgggggagtttgaac